(19) United States
(12) Patent Application Publication
(10) Pub. No.: US 2012/0264814 A1
(43) Pub. Date: Oct. 18, 2012

(54) Isolation, Cloning, Sequencing and Functional analysis of ss-casein promoter along with the regions of exon1, intron1 and exon2 using mammary gland derived cell line of Buffalo (Bubulus bubalis)

(75) Inventors: Subeer Suhash MAJUMDAR, New Delhi (IN); Nirmalya Ganguli, Burdwam (IN); Abul Usmani, Sagar Madhya Pradesh (IN)

(73) Assignee: NATIONAL INSTITUTE OF IMMUNOLOGY, New Delhi (IN)

(21) Appl. No.: 13/162,367

(22) Filed: Jun. 16, 2011

(30) Foreign Application Priority Data
Apr. 15, 2011 (IN) 1112/DEL/2011

(51) Int. Cl. C12N 15/85; A61P 31/04; A61K 48/00

(52) U.S. Cl. 514/44 R; 435/455

(57) ABSTRACT

The present invention relates to a method of in vitro isolation of buffalo β-caesin promoter (buCSN2) along with the regions of exon1, intron1 and exon2 from the genomic DNA in vitro (Bubalus bubalis) and its functional activity in using mammary cell line. The novel buffalo β-caesin promoter along with exon1, intron1 and exon2 is isolated and cloned upstream of the Enhanced Green flourescence protein (EGFP) gene and sequenced. The transfection of the DNA construct resulted into production of EGFP protein in mammary cell lines, confirming bioactivity of this newly isolated buffalo promoter sequence. More specifically, the present invention relates to isolation, cloning, sequencing and functional analysis of the buffalo β-casein promoter in vitro using mammary cell line.

### CROSS REFERENCE

The following application claims the benefit of and priority from Indian Patent Application Number 1112/DEL/2011 filed Apr. 15, 2011 which is incorporated herein by reference.

### FIELD OF THE INVENTION

The present invention relates to a method of in vitro isolation of β-caesin promoter (buCSN2) along with the regions of exon1, intron1 and exon2 from the genomic DNA of Bubalus bubalis and accessing and/or determining the functional activity thereof using mammary cell line. A novel buffalo β-caesin promoter is isolated, cloned, sequenced in vitro and transfected in the mammary cells thereby confirming the functionality of the newly isolated buffalo promoter sequence.

### BACKGROUND OF THE INVENTION

The easy automation of DNA sequencing has greatly facilitated the characterization of genes associated with the milk proteins in various species. Several milk protein genes, primarily from rodents or dairy animals, have been cloned and sequenced, but knowledge of the genes encoding buffalo milk proteins is still sparse.

Over the past decades, researchers, using biotechnology, developed systems which demonstrated promoter elements with tissue specificity driving foreign DNA to express products, (Reddy, 1991). However only a limited number of promoter elements have actually been introduced into domestic farm animals. So far, many promoters have been isolated and used to drive the expression of foreign gene in the mammary gland of goats, pigs and cows etc. However no use of buffalo specific promoter has been done in the past for milk gland specific expression and secreation of recombinant protein in the mammary gland cells. The presently available buffalo β-casein promoter sequences, which are given in fractions National Center for Biotechnology Information (NCBI) nucleotide data base, are unable to express the exogenous protein in the mammary gland in a secretory form as all of them lack the Exon2 which contains necessary signal peptide sequence for secretion in the milk. Therefore, there is need for such strong promoter sequences, which can drive the complete expression and secretion of foreign gene specifically in the buffalo mammary gland.

Buffalo milk is composed of approximately 88% water, 3.3% protein and the remaining portion comprises carbohydrates and fat. The caseins, comprising 80% milk protein, are divided into four groups, alpha S1, alpha S2, beta and kappa casein. Casein genes are expressed in a tissue-specific and highly coordinated manner. The main goals of casein gene promoter studies are to unravel cis- and trans-acting factors involved in the complex signalling pathway controlling milk production, and to explore the possibility of using these promoters for tissue-specific production of heterologous proteins in the mammary gland. Casein genes are clustered on the chromosome 6 (BTA 6) within the region exceeding 200 kb, of the casein locus cluster (RIJNKELS et al., 1997; FERRETTI et al., 1990), containing three paralogs encoding the calcium-sensitive αₛ₁-(CSN1S1), α₅₂—(CSN1S2) and β-(CSN2) casein (BONSING and MACKINLAY, 1987), as well as an evolutionary unrelated κ-(CSN3) casein gene. The relative concentration of calcium sensitive caseins and x-casein in the mammary gland is affected by casein genetic variants and has a significant impact on micelle size and technological properties of milk (LODES et al., 1996; GERNAND and HARTUNG, 1997; JUSZCZAK et al., 2001).

β-casein (CSN2) is the most abundant protein in milk and is expressed in a higher concentration in buffalo milk. The specific expression of this protein is, therefore, of crucial importance for the advancement of milk production. The most studies dealing with the expression of β-casein gene in the cell culture systems and transgenic mammals had been performed in other bovine species particularly cows and goats. However, nothing similar has yet been demonstrated in buffalo (Bubulus bubalis) where a promoter can drive the expression of any exogenous protein in the mammary gland in a secretory form as all of the buffalo CSN2 promoters available so far lack the Exon2 which contains necessary signal peptide sequence for secretion in the milk.

Since buffalo udder has capacity to produce more protein in the milk as compared to cow or goat udder, the identification and isolation of important promoter region of the buffalo β-casein (JANN et al., 2002) would provide an enhanced opportunity for producing therapeutic proteins in buffalo milk.

In fact, until now most therapeutic proteins have been produced by cell culture systems, which use cells such as yeast, bacteria or animal cells. However, it is difficult to produce proteins in large scale using cell culture systems because of its limited capacity and high cost. Furthermore, for some in vitro produced proteins, additional steps are required to introduce proper post-translational modifications such as glycosylation, γ-carboxylation, hydroxylation and so on (Houdebine et al., Transgenic Res., 9(4-5); 305-320, 2000; Lubo et al., Transgenic Res., 9(4-5); 301-304, 2000). Animal bioreactors that produce valuable or therapeutic proteins have been evaluated as efficient and cost-effective expression systems. In particular, the large-scale production of therapeutic recombinant proteins from transgenic animals is much more cost-effective and biologically efficient compared to the cell culture system (van Berkel et al., Nat. Biotechnology., 20(5); 484-487, 2002). Therapeutic proteins produced in animal milk were known to be post translationally modified in a way very similar to the human counterpart proteins (Edmunds et al., Blood, 91(12); 4561-4571, 1998; Velander et al., Proc Natl Acad Sci USA., 89(24); 12003-12007, 1992; van Berkel et al, Nat. Biotechnol., 20(5); 484-487, 2002). The mammary gland can express more than 2 g of heterologous recombinant proteins per liter of milk (Velander et al., 1992; van Berkel et al., 2002). Based on the assumption of average expression levels, daily milk volumes and purification efficiency, 100 goats for 100 kg of monoclonal antibodies, 75 goats for 75 kg of antithrombin III and two sows to produce 2 kg of human clotting factor IX is enough to fetch worldwide requirement per year (Rudolph, 1999). Therefore, a small herd of transgenic livestock could supply the world demand for pharmaceuticals, which cannot be expressed by other systems such as bacteria or fungi, mainly due to limitation in complex post-translational processing which is necessary for their proper biological function. Therefore, a small herd of transgenic livestock could fulfill the world demand for pharmaceuticals, which cannot be expresses by other systems such as bacteria of fungi, mainly due to the necessity of complex post translational processing to ensure their proper function.

Taking all these things into consideration, the present invention is directed to method of isolating and cloning specific promoter sequences of β-casein gene along with the regions of exon1, intron1 and exon2, which may drive the expression of every foreign gene specifically in the mammary gland. The purpose of the present invention is to isolate β-caesin promoter from the blood of buffalo (Bubalus bubalis) in vitro and access and/or determine the biological activity of said promoter in cultured mammary gland cells. The buffalo β-caesin promoter is sequenced after isolation and this unique promoter is cloned upstream of the gene for Green Fluorescence Protein (GFP). The transfection of the construct results into expression of GFP in cell lines from mammary glands confirming functionality of this newly isolated buffalo promoter sequence. The newly invented buffalo β-casein promoter is very strong and tissue specific promoter, which contributes in the progress and development of the mammary gland expression for the production of valuable proteins in the mammary cell lines. Extrapolation of this invention may be done in future to generate therapeutic proteins in the milk of buffalo by specific expression of genes, related to proteins of human use cloned under the present invented β-caesin promoter, in the cells of the mammary gland.

### SUMMARY OF THE INVENTION

The present invention, therefore, relates to a method of in vitro isolation of buffalo β-caesin promoter (buCSN2) along with the regions of exon1, intron1 and exon2 from the genomic DNA in vitro (Bubalus bubalis) and its functional activity in using mammary cell line. The novel buffalo β-caesin promoter along with exon1, intron1 and exon2 is isolated and cloned upstream of the Enhanced Green flourescence protein (EGFP) gene and sequenced. The transfection of the DNA construct resulted into production of EGFP protein in mammary cell lines, confirming bioactivity of this newly isolated buffalo promoter sequence.

Accordingly, the present invention relates to isolation, cloning, sequencing and functional analysis of the buffalo β-casein promoter in vitro using mammary cell line.

In an alternative embodiment, the present invention provides an effective way to generate therapeutic proteins in mammary gland cells for large scale production using the invented buffalo β-caesin promoter.

### BRIEF IDENTIFICATION OF DRAWINGS

The patent or application file contains at least one drawing executed in color. Copies of this patent or patent application publication with color drawing(s) will be provided by the Office upon request and payment of the necessary fee.

FIGS. 1 (a) to 1 (f) depict Construct Making Details of pbuCSN2-IRES2-EGFP

a) Gel picture showing amplicon (˜3.8 kb) of Buffalo genomic fragment containing β-casein promoter, Exon1, intron1 and Exon2 (obtained by long PCR from Buffalo Genomic DNA) visualized in 0.8% agarose gel;

b) 0.8% agarose gel showing fragment of (˜5.5 kb) pCMV-Sport6 vector linearized by SmaI restriction enzyme;

c) restriction digestion analysis of vector pCMV-SPORT6-buCSN2 by AhdI showed right digestion pattern (red circle) in 2 clones, after ligation;

d) Linearization of pIRES2-EGFP vector by restriction digestion using PstI and XmaI;

e) Restriction digestion of pbuCSN2-IRES2-EGFP vector backbone using PstI after ligation;

f) Restriction digestion of pbuCSN2-IRES2-EGFP vector with PstI and SfiI after ligation; Note that the fragment of ˜6.5 kb (containing buCSN2promoter+Exon1+Intron1+Exon2-IRES2-EGFP) was eluted and used for in vitro transfection.

FIG. 2 depicts Vector Map of pbuCSN2-IRES2-EGFP construct

FIG. 3 depicts:

Sequencing performed by Chromosome Walking method from both the end of the insert. Forward primer (BC_SSM_{—}170: 5′ GATTTCCAAGTCTCCACCC 3′; SEQ ID NO 1) was designed on CMV promoter sequence in the 5′ side and reverse primer (BC_SSM_{—}236: 5′ ATATAGACAAACGCACACCG 3′; SEQ ID NO 2) was designed on the IRES sequence in the 3′ side of the buCSN2 promoter.

FIGS. 4 (a) and 4 (b) depict:

(a) Alignment results of buffalo CSN2 Promoter region along with exon1, intron1 and exon2 with cow. Note that the alignment was performed using EMBL-EBI EMBOSS Pairwise Alignment Algorithms.

b) Comparative analysis of sequence of buffalo CSN2 genomic fragment (containing buCSN2promoter, Exon1, Intron1 and Exon2) with various sequences of buffalo CSN2 promoter available in National Centre for Biotechnology Information (NCBI) till date. Different line fragments represent different accession numbers as elaborated hereafter.
Y17836.1: Match from 11 bp to 321 bp with Newly Invented buCSN2 sequence (11 mismatch, 1 gap)
Y17838.1: Match from 1535 bp to 1737 bp with Newly Invented buCSN2 sequence (3 mismatch, 0 gap)
GQ176291.1: Match from 1400 bp to 1750 bp with Newly Invented buCSN2 sequence (so many mismatches and gaps)
GQ259485.1, GQ259484.1., GQ259483.1, GQ259482.1, and GQ176290.1: Match from 47 bp to 1774 bp with Newly Invented buCSN2 sequence (somany mismatches and gaps).
AY352050.1: Match from 1106 bp upstream to 3211 bp of Newly Invented buCSN2 sequence (so many mismatches and gaps).
FM986648.1: Match from 275 bp to 1789 bp of Newly Invented buCSN2 sequence (5 mismatches, 15 gaps)

FIGS. 5 (a) to 5 (c) depict in vitro transfection of linearized fragment of pbuCSN2-IRES2-EGFP in MCF-7 cells (mammary gland carcinoma cell line).

a) phase contrast image of transfected MCF-7 cells.

b) Fluorescent image of transfected MCF-7 cells under UV light. Fluorescent expression EGFP can be seen in few cells.

c) Merged image of the transfected MCF-7 cells

### DETAILED DESCRIPTION OF THE INVENTION

Prior to setting forth the invention in detail; it may be helpful in the understanding thereof to define the following terms.

Genes—A length of DNA that carries the genetic information necessary for production of a protein. Genes are located on chromosomes and are the basic units of heredity.

Promoter—It is a controlling element in the expression of the gene. It serves as a recognition signal for an RNA polymerase and marks the site of initiation of transcription. A promoter is a region of DNA that facilitates the transcription of a particular gene.

Transgenic animal—A transgenic animal is one that carries a foreign gene that has been deliberately inserted into its genome. The foreign gene is constructed using recombinant DNA methodology.

Recombinant protein—a protein or peptide coded for by a DNA sequence which is not endogeneous to the native genome of the mammal in whose milk it is produced in accordance with this invention or a protein or peptide coded for by a DNA sequence which if endogeneous to the native genome of the mammal in whose milk it is produced does not lead to the production of that protein or peptide in its milk at the same level that the transgenic mammal of this invention produces that protein in its milk.

Therapeutic proteins—Proteins that are engineered in the laboratory for pharmaceutical use are known as therapeutic proteins. The majority of biopharmaceuticals marketed to date are recombinant therapeutic protein drugs.

A preferred embodiment of the present invention relates to method steps comprising—

a) Isolating the genomic fragment containing the buCSN2 (buffalo β-caesin) promoter region along with the regions of exon1, intron1 and exon2 from the blood of buffalo in vitro
b) Cloning the isolated genomic fragment into pCMV-SPORT6 vector to develop pCMV-SPORT6-buCSN2.
c) Further sub cloning of buCSN2 from pCMV-SPORT6-buCSN2 into pIRES2-EGFP vector (Clonetech, USA) to develop the pbuCSN2-IRES2-EGFP plasmid.
d) Sequencing and confirmation of the clones of step c)
e) In vitro transfection of the linearized DNA construct of step c) into cell line MCF7 cell line derived from the mammary gland.

In an alternative embodiment, where the in vitro transfected cells express the desired protein in the mammary gland cells, there is envisaged a means of detection of expressed protein include the florescent observation of EGFP under UV light using TE2000-S microscope (Nikon) fitted with epi-fluorescence attachment.

To test the potential usefulness of the isolated buffalo β-caesin promoter in expressing recombinant protein, the buffalo β-casein promoter was cloned and tagged using Enhanced Green Fluorescent Protein (EGFP). It was ascertained that the presently invented buffalo β-caesin promoter facilitates mammary gland-specific expression and secretion of any protein. Therefore, a promoter of the present invention using the same promoter may enable secretion of the therapeutic proteins in milk, which consequently will provide benefits for the production of useful proteins that are medically and pharmaceutically valuable.

Therefore, the newly invented buffalo β-casein promoter along with exon1, intron1 and exon2 is very strong and tissue specific promoters, which may contribute in the progress and development of the mammary gland expression for the production of valuable proteins in the milk.

For the purposes of illustration, the invention will be described non-limitatively in the following examples. The following examples are provided in order to demonstrate and further illustrate the preferred embodiments and aspects of the present invention. It should be understood that such embodiments are by way of example only and merely illustrative of a small number of many possible embodiments which can represent applications of the principles of the present invention.

### Example 1

### Bioinformatics

### Annotation and Primer Designing

As Indian river buffalo (Bubalus bubalis) genome is not fully annotated so there was no Genomic contig sequence available which can be used as a reference for designing the primer to isolate the buffalo β-casein (buCSN2) promoter region. buCSN2 cDNA sequence had been published previously by Garg et. al. This sequence was compared with Cow (Bos taurus) genomic contig sequence for annotation. This analysis showed a significant match throughout the cDNA region as both the species are from same family (Bovine). Four different forward primers were designed with fixed reverse primer (which sits on Exon2). These primers result in PCR products with different amplicon sizes viz., 7 kb, 4.2 kb, 3.8 kb and 2.3 kb.

### Example 2

### Genomic DNA Isolation

To obtain a good quality and high yield of genomic DNA, isolation was performed from blood (collected aseptically from buffalo) using blood gDNA isolation kit (Advance Micro Devices, India), dissolved in TE buffer (10 mM Tris, 0.1 mM EDTA. pH 7.8) and stored at 4° C.

### Example 3

### Long PCR for the Isolation of Buffalo β Casein Promoter Region Along with Exon1, Intron1 and Exon2

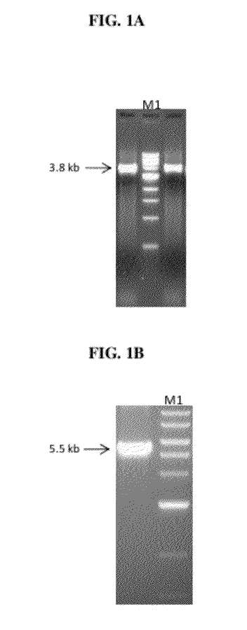

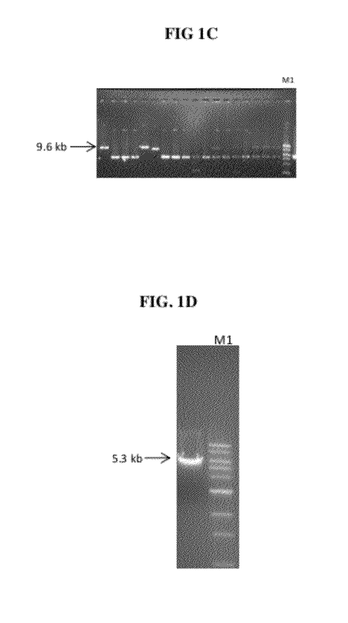

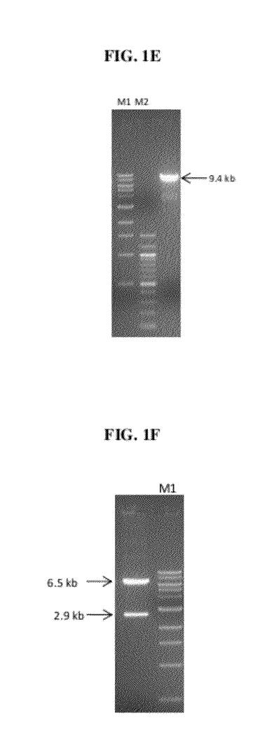

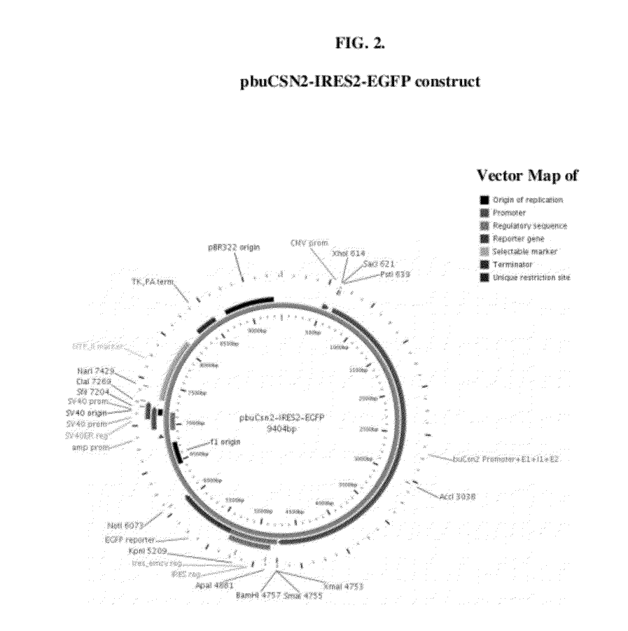

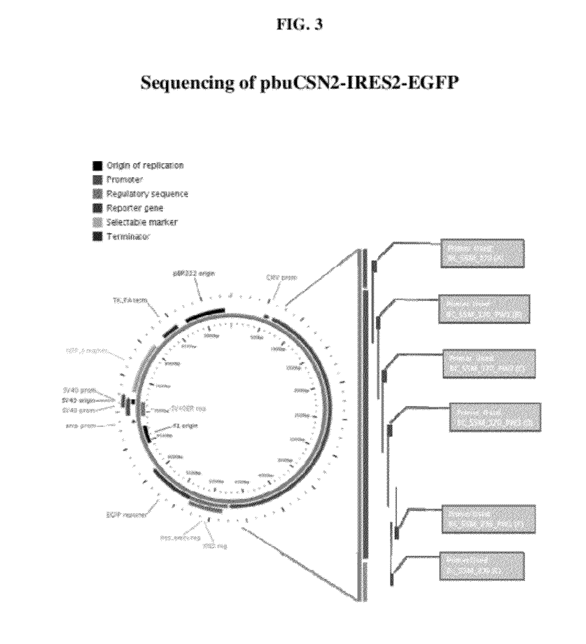

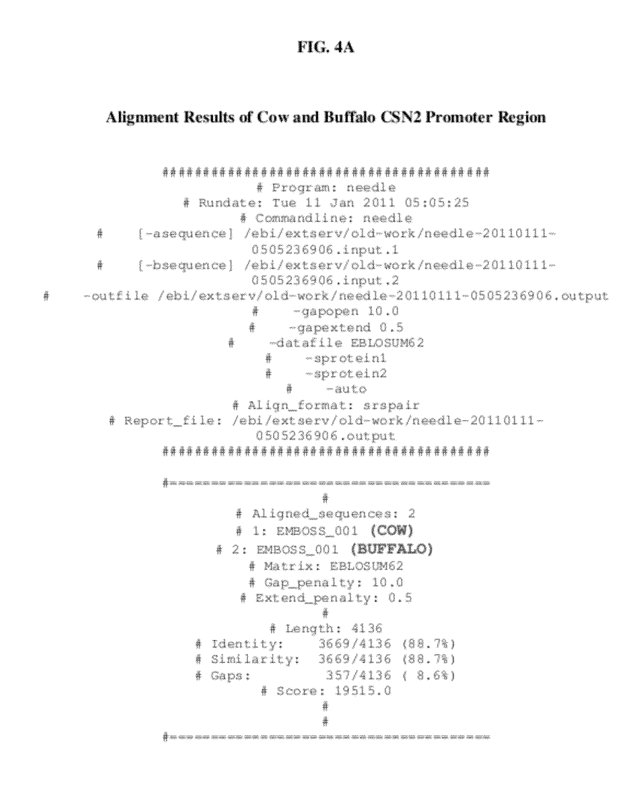

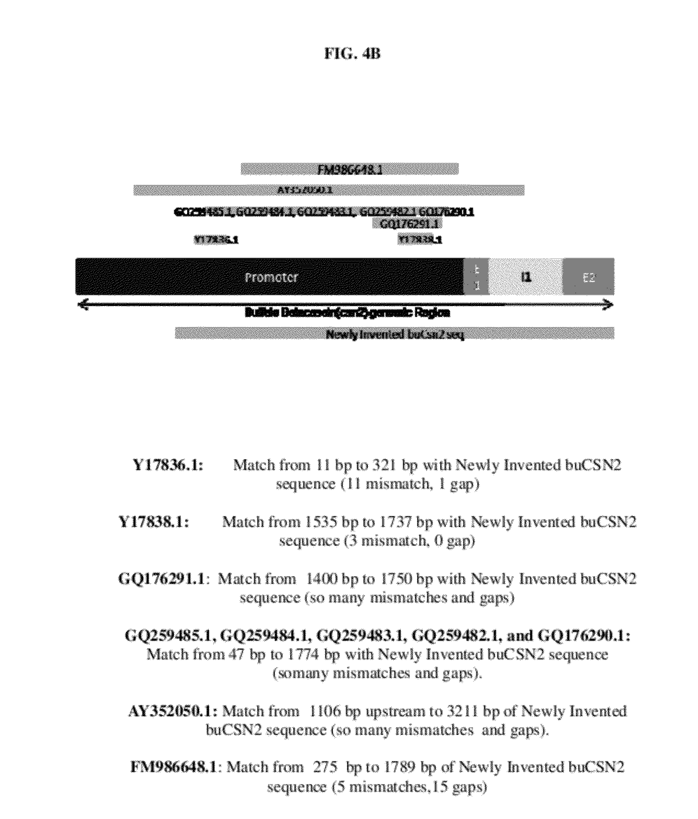

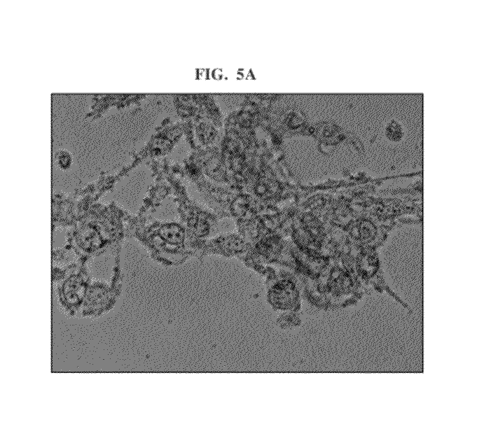

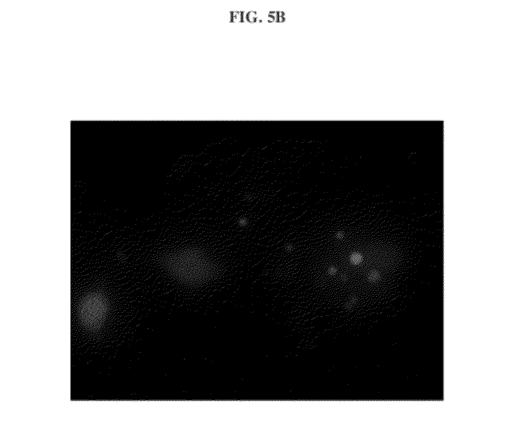

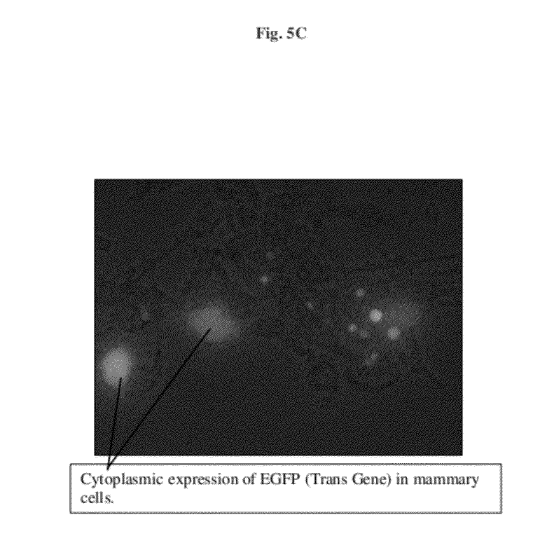

Long PCR protocol was used for isolating the genomic fragment containing the buCSN2 (buffalo β-casein) promoter region along with exon1, intron1 and exon2. PCR was carried out in a Biorad Thermal Cycler (S1000) using PCR reaction mix comprising 100 ng gDNA, 1.5 unit Pfu DNA polymerase with 3′ to 5′ proof reading activity (Fermentas, USA), 4.0 mM Mg⁺², 0.25 mM dNTP's, 1×Pfu reaction buffer and 2.5 μM of each primer (Table 1) in a 10 μl reaction using specific thermal cycling parameters (FIG. 1a).

TABLE 1
Buffalo CSN2 Long PCR Primer Set

| | |
|---|---|
| Forward Primer | GCCTGCAGTCTGGTCCAATCGAATCCATCTC |
| | (SEQ ID NO 3) |
| Reverse Primer | GCCCCGGGTATTTACCTCTCTTGCAAGGGCC |
| | (SEQ ID NO 4) |

Thermal Cycling parameters

| Step1 | | | | | Step6 |
|---|---|---|---|---|---|
| Initial | Step2 | Step3 | Step4 | Step5 | Final |
| Denaturation | Denaturation | Annealing | Extension | Repeat | Extension |
| 94° C. for | 94° C. for | 66° C. for | 72° C. for | From step2 | 72° C.for |
| 4 min | 45 sec | 45 sec | 4 min | for 29 | 10 min |
| | | | | cycles | |

### Example 4

### Cloning and Sub-Cloning of Genomic Fragment Containing the buCSN2 in the Expression Vector

The amplicon was eluted from Agarose gel using Gel Extraction kit (Qiagen, Germany) procedure. The eluted sample was checked on 0.8% agarose gel for confirmation. The ˜3.8 kb amplicon obtained from long PCR was cloned into pCMV-SPORT6 vector, which was priorly linearized with SmaI (resulting in blunt ended vector backbone). The amplicon and vector were treated with T4 Poly Nucleotide Kinase and Antarctic phosphatase respectively, followed by blunt end ligation using T4 DNA Ligase to develop pCMV-SPORT6-buCSN2. Clones harbouring right insert were detected by AhdI restriction enzyme digestion. The desired insert was subsequently sub cloned into pIRES2-EGFP vector (Clontech, USA) to develop the pbuCSN2-IRES2-EGFP plasmid. In this subcloning process both the vector was digested with PstI and XmaI. This strategy positions the insert in a 5′→3′ orientation in pIRES2-EGFP vector backbone, resulting the expression of EGFP being regulated through buCSN2 promoter. Clones thus obtained were checked by linearising the pbuCSN2-IRES2-EGFP with PstI that resulted into 9.4 kb fragment and were further confirmed by sequencing. pbuCSN2-IRES2-EGFP vector was digested with PstI and SfiI and the fragment of ˜6.5 kb (containing buCSN2promoter+Exon1+Intron1+Exon2-IRES2-EGFP) was eluted and used for in vitro transfection study. The whole strategy of this cloning has been shown in FIG. 1a-f).

### Example 5

### Sequencing of pbuCSN2-IRES2-EGFP

Sequencing of pbuCSN2-IRES2-EGFP was performed by Chromosome Walking method from both the ends of the insert. Forward primer (BC_SSM_{—}170: 5′ GATTTCCAAGTCTCCACCC 3′; SEQ ID NO 1) was designed on CMV promoter sequence in the 5′ side and reverse primer (BC_SSM_{—}236: 5′ ATATAGACAAACGCACACCG 3′; SEQ ID NO 2) was designed on the IRES sequence in the 3′ side of the buCSN2 promoter (FIG. 3). As shown in the Appendix, different Sets (SET A, B, C, D, E, F) represents single pass sequencing output using various sets of primers. Full and complete sequence of buffalo CSN2 promoter region along with exon1, intron1 and exon2 is shown in the Appendix FIG. 3c.

The obtained sequence of Buffalo CSN2 Promoter Region was aligned with the sequence of cow CSN2 promoter region and alignment results of buffalo CSN2 Promoter region along with exon1, intron1 and exon2 with coware shown in FIG. 4a. The alignment was performed using European Molecular Biology laboratory align 2.0 software.

### Example 6

### Comparative Analysis of Sequence of Buffalo buCSN2 Genomic Fragment with Various Sequences of Buffalo CSN2 Promoter

Comparative Analysis of sequence of newly invented buffalo CSN2 genomic fragment (containing buCSN2promoter, Exon1, Intron1 and Exon2) was done with various sequences of buffalo CSN2 promoters available in National Centre for Biotechnology Information (NCBI) till date (FIG. 4b). The above analysis showed that all the presently available sequences are unable to express the exogenous protein in the mammary gland in a secretory form as all of them lack the Exon2 which contains necessary signal peptide sequence for secretion in the milk

### Example 7

### Functional Analysis of pbuCSN2-IRES2-EGFP (In-Vitro Transfection of pbuCSN2-IRES2-EGFP in the Mammary Gland Derived Cell Line)

This pbuCSN2-IRES2-EGFP construct was linearised by double digestion using PstI and SfiI restriction enzyme and eluted using gel extraction kit (Qiagen, Germany) before transfection into the mammary gland derived cell lines. MCF-7 cell line (mammary gland carcinoma cell line) was used for In-vitro transfection of pbuCSN2-IRES2-EGFP. The cells were electroporated with linearized DNA construct of pbuCSN2-IRES2-EGFP and cultured at 39° C. in CO₂ incubator (5% CO₂ in air). EGFP expression was observed after 3 days of electroporation. Cells were maintained up to 14 days to ensure the expression of the EGFP from the transgene which has been integrated into the genome. This protein expression confirmed that the buffalo β-casein promoter is biological active and can drive the expression of any foreign gene tagged with it.

Those skilled in the art will recognize or be able to ascertain using no more than routine experimentation, many equivalents to the specific embodiments of the invention described herein. Such equivalents are intended to be encompassed by the following claims.

### REFERENCES OF CITED DOCUMENTS

Reddy V. B. Expression of human growth hormone in the milk of transgenic mice. Animal Biotechnology. Animal Biotechnology, 2(1), 15-29 (1991).
Organization of the bovine casein gene locus M. Rijnkels, P. M. Kooiman, H. A. de Boer and F. R. Pieper Mammalian Genome Volume 8, Number 2, 148-152,
RIJNKELS, M.; WHEELER, D. A.; DE BOER, H. A.; PIEPER, F. R.: Structure and expression of the mouse casein gene locus. Mammalian Genome 8 (1997), 9-15
FERRETTI, L.; LEONE, P.; SGARAMELLA, V.: Long range restriction analysis of the bovine casein genes. Nucleic Acids Res. 18 (1990), 6829-6833
BONSING, J. MACKINLAY, A. G.: Recent studies on nucleotide sequences encoding the caseins. J. Dairy Res. 54 (1987), 447-61
LODES, A.; KRAUSE, I.; BUCHBERGER, J.; AUMAN, J.; KLOSTERMEYER, H.: The influence of genetic variants of milk proteins on the compositional and technological properties of milk. 1. Casein micelle size and the content of non.glycosylated x-casein. Milchwissenschaft 51 (1996), 368-373
GERNAND, E.; HARTUNG, H.: Untersuchungen zu Einflussgröβen auf Zusammensetzung und Käsereitauglichkeit von Rohmilch einzelner Kühe. 2. Mitt.: Untersuchung zur Variation der Milchgerinnung und deren Ursachen an Einzelmilchproben. Arch. Tierz., Dummerstorf 40 (1997) 3, 225-238
JUSZCZAK, J.; ERHARDT, G.; KUCZAJ, M.; ZIEMTNSKI, R.; PANICKE, L.: Zusammenhang zwischen κ-Casein und β-Lactoglobulin-Varianten mit der Milchleistung und der Nutzungsdauer von Rindern der Rassen Schwarzbuntes Rind und Polnisches Rotvieh. Arch. Tierz., Dummerstorf 44 (2001) 3, 239-249
JANN, O.; PRINZENBERG, E.-M.; BRANDT, H.; WILLIAMS, J. L.; AJMONE-MARSAN, P.; ZARAGOZA, P.; ÖZBEYYAZ, C.; ERHARDT, G.: Intragenic haplotypes at the bovine CSN1S1 locus. Arch. Tierz., Dummerstorf 45 (2002) 1, 13-21
Houdebine L M (2000). Transgenic animal bioreactors. Transgenic Res. 9:305-320.
Lubo et al., Transgenic Res., 9(4-5); 301-304, 2000)
VAN BERKEL, Patrick H. C.; WELLING, Mick M.; GEERTS, Marlieke; VAN VEEN, Harry A.; RAVENSBERGEN, Bep; SALAHEDDINE, Mourad; PAUWELS, Ernest K. J.; PIEPER, Frank; NUIJENS, Jan H. and NIBBERING, Peter H. Large scale production of recombinant human lactoferrin in the milk of transgenic cows. Nature Biotechnology, May 2002, vol. 20, no. 5, p. 484-487
T. Edmunds, S. M. Van Patten, J. Pollock, E. Hanson, R. Bernasconi, E. Higgins, P. Manavalan, C. Ziomek, H. Meade, J. M. McPherson, et al. . . . Transgenically Produced Human Antithrombin Structural and Functional Comparison to Human Plasma-Derived Antithrombin Blood, 91(12); 4561-4571
Velander, W. H., Page, R. L., Morcol, T., Russell, C. G., Canseco, R., Drohan, W. N., Gwazdauskas, F. C., Wilkins, T. D. & Johnson, J. L. (1991) Ann. N.Y. Acad. Sci. 665, 391-403.
Rudolph, N. S., 1999. Biopharmaceutical production in transgenic livestock. TIBTECH 17, 367-374.

APPENDIX
>BC_SSM-170 (SET A)
GAGCTGGGGGTAGTTTGGCACCAAAATCAACGGGACTTTCCAAAATGTC
GTAACAACTCCGCCCCATTGACGCAAATGGGCGGTAGGCGTGTACGGTG
GGAGGTCTATATAAGCAGAGCTGGTTTAGTGAACCGTCAGATCCGCTAG
CGCTACCGGACTCAGATCTCGAGCTCAAGCTTCGAATTCTGCAGTCTGG
TCCAATCGAATCCATCTCTATCAATTAATATAATTCAAAATTGGTGAGA
GACAGTCTTAAGGAAATGCTGTGTTTATTGCACAATATGTAAAGCATCT
TCCTGAGAAAAGGGAAATGTTGAATGGGAAGGACATGCTTTATTTTGTA
TTCCTTTTCTCAGAAATCACACTTTTTTGCCTGTGGCCTTGGCAACCAA
AAGCTAACACATAAAAAAAGGCATATGAAGTAGCCAAGGCCTTTTCTAG
TTATATCTATGACATTGAGTTCATTTTATCATTTATTTTCCTGACTTCC
TCCTGGGTCCATATGAGAATCTTAGAATGAATATTAGCTGAAAATCCAA
ATACATAGTAGATGTTGATTTGGGTTTTCTAAGCAATACAAGACTTGTA
TGACAGTGAGATGTATTACCATCCAACACACATCTCAGCATGATATAAA
CGTAAGGTATATTGTGAAGAAAAATGATCAATTATGTCAAAGTGCTTAC
TTTAGAAGATCATCTATCTGTCCCAAAGCTGTGAATATATATATTGAAT
GTAATGAATAGATGAAACAAACCTTGTAAAAATGAGTAGTGTAAAATAC
AACTACATTTATGAACATCTATCACTAAAGAGGCAAAGAAAGTTGAGGA
CTGCTTTTGTAATTGGGCTCCTATTAATAAAAAGTACTTCTGAGGTCTG
GCTTAGACTCTATTGTAGTACTTATGGTAAGACCCTCCTCCTGTCTGGG
CTTTCATTTTCTTCTTCCTTTCCCTCATTTGCCCTTCCATGAATACTAG
CTGATAAACATTGGCTCACTATAAAGATATGAGCAAACTGAGCTGTCCA
TTTATAAATCTGTATAAAATAAATATTTTGTTCTTACAGGAAGTATTTT
CCTCTTA (SEQ ID NO 5)
>BC_SSM_170_PW_1 (SET B)
TATTGACCCCTTTGTTTTGGGCCTCCTTATTAATAAAAAGTACTTCTGA
GGTCTGGCTTAGACTCTATTGTAGTACTTATGGTAAGACCCTCCTCCTG
TCTGGGCTTTCATTTTCTTTCTTCCTTCCCTCATTTGCCCTTCCATGAA
TACTAGCTGATAAACATTGGCTCACTATAAAAGATATGAGGCCAAACTT
GAGCTGTCCCATTTTAATAAATCTGTATAAATAATATTTGTTCTACAGA
AGTATTCTCTAAATAAATGTTACTTTCTCTCTTAAAATCCCTCAACAAA
TCCCCACTATCTAGAGAATAAGATTGGCATTCCCTGGAGTCACAGCATG
CTTTGTCTGCCATTATCTGACCCCTTTCTCTTTCTCTCTTCTCACCTCC
ATCTACTCCTTTTTCCTTGCAATTCATGACCCAGATTCACTGTTTGATT
TGGCTTACATGTGTGTGTGCTGAGTTGTGTCTCACTCTTGTCAACCCCA
TGAATGATAGTCCACCAGGCTCCAGTGTCCATGAAATTTTCCAGTCAAG
AATGCTGGAGTGGATTGCATTTCCTACTCCATTTGATTAATTTAGTGAC
TTTTAATTTTTTTTTCCCTCCATATTCAGGAGGCTATTCTTTCCTTTTA
GTCTATACTGTCTTTGCTCTTCAGGTCTAAGCTATCATCATGTGCTTGT
TAGCTTGTTTCTTTCTCCATTATAGCATAACACTAACAACTATTCAGGT
TAGCATGAGATTGTGTTCTTTGTGTGGCCTGTGTATTTTCTGGGTGGTG
TATTAGAATTACCCTAGATCTCAAAGGACCCACCGAATACTAAAGAAGA
CCTCATTGTGGTTACATATTTGGGGACTCGGTCAAAACTTCGCATATCC
AGCAAAGTCTGTAGCTACTGGACAAATTGACTTTCCATTTATCGAATTG
TGACTCATCCATTGAAATGCCTCCTGATCGGAACGAGAATACGGAGGAC
(SEQ ID NO 6)
>BC_SSM_170_PW_2 (SET C)
AAAACCATCAAGACCCCCACCCGAATACTAAAAGAGACCTCATTGTGGT
TACAATAATTTGGGGACTCGGCCAAAACTTCCGCATATCCCAGCCAAGG
TCTGTAGCTACTGGACAATTTAATTTCCTTTATCAGATTGTGAATTATT
CCCTTTAAAATGCTCCCCAGAATTTCTGGGGACAGAAAAATAGGAAGAA
TTCATTTTCTAATCATGCAGATTTCTAGGAATTCAAATTCACTATTGGT
TTTATTTCAAACCACAAAATTAGCATGCCATTAAATACTATATATAAAC
AGCCACAAAATCAGATCATTATCCATTCAGCTCCTCCTTCACTTCTTGT
CCTCTACTTTGGAAAAAAGGTAAGAATCTCATATATAATTTCAGTGTAT
CTGCTACTCATCTTTATTTCAGACTAGGTTAAAATGTAGAAAGAACATA
ATTGCTTAAAATAGATCTTAAAAATAAGGGTGTTTAAGATAAGGTTTAC
ACTATTTTCAGCAAATTTGTTAAAAAATAGAAGCAACTATAAAGATTTG
ATAAAAATTATAGTGACTGCAAATGTTTTAGGAATATAATAAGATGTAG
TAACAGTGGTTGCTATTTTCTTTACCACGAGACTAGTTAACAGGCTGTA
TTAAAAGATCTTTTCTTGAATTAAATATTTTCAATTTGATTAAACCTAT
CTCAGCCATAAAGGCAAGCACATTTAATTTATACTATGGGGATTTGAAA
TAATTATTACTGAAGAAGCTCTACCAAACAAAAAGTTTATAGAGCTAGC
AGAGGGAGAGGGTGGGATGATTTGGGGAGAATGACATTGAAACATGTAA
TAATATCATGTATGAAATGAGTCGCCAGTCCACGTTCGATGCCACGATA
CTGCATGCTGGGGCTGTGCCACTGGACGACCAGAGGATGTATGGAGAGA
GGGAAAGAAGGTTCCAGAATGGAACCGTTATACCCGTGCGGAATCATCC
TAAATTGGCAAAC (SEQ ID NO 7)
>BC_SSM_170_PW_3 (SET D)
GGTACCCGGTTATTTATTCATGGTATGAATGAGTCGCCAGTCCACGTTC
GATGCACGATACTGGATGCTTGGGGCTGGTGCACTGGGACGACCCAGAG
GGATGGTATGGGGAGGGAGGAGGGAGGAGGGTTCAGGATGGGGAACACG
TGTATACCTGTGGCGGATTCATTTCAATATTTGGCAAAACCAATACAAT
ATTGTAAAGTTTAAAAATAAAATAAAATTAAAAAAAAAGATAAAGAGGG
TTGTTAGGATACATCTGCTATTTGAAAGGTGTTTATAAAAGAAGAGTAT
ATTTATCAAAATTGCTCAGAACATCCAAATTTCAAGTTTATCATTTATC
TTACAATATTTCAAAAATATTAAAATAAATACATGAAATACAGAAGTAA
ATTAAAGAGAAAGTATTTTATTTTGTAAAAAAAATTATAGGTTGGATAG
GGAGTACCAGGAAACAAAAAACAATGAAAAATGTGATCTGACAGAAATT
ATAGCTCAAAGTATAGTAGTCAGTAATGAAATGGCTTAAAAATTGGCAT
ATAAAATGCTAATTATAAAATAAACAAAATATAATAATATCCTCCCTAC
ATGTAATGAACTCTGAGTATTATACTCTTTTTTTGAAGTCTTGACAATA
AAAATTTATTTAAGAAGTTTATAGACATCTTGGATAAAGTAAAACAAAC
TAAGAATTAGCATCCATGAGAAAAATATAGAAAAAATTTCTTTAATTTA
GTTTGCAAAATCTGGGATTGAAGAATGTGTGTTAAGCAGATGTTTGATG
GGCAAGGAACCATTTTTGTTTTCAAAGAAGCTGTATGAAAAATGCAACA
ACAACCTATTTTAATTACATTTTGTCAAAAAACAATATGTATTTTAATT
TATGCCTAACAGGGAGAAAGTTAGTCTAAGTGGGGACTGGCAAGAGAAA
TCCTCACACCCCTGGTAATCACAAGAGAAATAGTTACACCAGTCTCTGT
AAGATAGGCTAAGTTACGATTCTCTTACA (SEQ ID NO 8)
>BC_SSM-236 (SET E)
CTTTAGCCGGCTTCGGGCCAGTAAACGTTAGGGGGGGGGGAGGGAGAGG
GGCGGATCCCGGGTATTTACCTCTCTTGCAAGGGCCAGAGCCACCAGGC
AGGCAAGGATGAGGACCTTCATGGCTCTCAATTTCTGTGAATGGGAAGA
TGAGGAAACAGTGTATTACTTGTCAATCTATTTTCTTATTTAAGTAGAA
TTACTTCCCTTTAAAAGAAACCAAAGATAGTGTATAATACAAATGTCCA
AGACTCCATGCTTCCAGGGGAGCGCAGGTTTGATCCCTCGTCAGGGAAC
CAAGATCCTGCAAACCTCATGGCAAAAACAAAAAGCCCTGTATTTTAGA
AAAATATTAATAGACATTTTATCATATAGCCAAGGTTAAAATTTACTAT
TACTCATTAATCTTACATTTCAAGTTAATGTTTCACTGCCAGCCATTCA
GATGTTTTCAGCTAACAGCCTATATCTTATTCCACTCTTTTTCATCTTA
TTCACAGTACTTATGAGTGTTTTTTGGAAAGACAGAAGTTACAGATATT
TACAGAAAAATTAAGTGATTGATTCACTTATTAAAAAATATTTATTTAT
TAATTTTTATTGCCAGATATTGCTTAGATTCTGGGATTAGAGCAGTGAA
CAAAATATTAAGGTTTCAGTTCTATTGGAGACTCAGTTTTATTGAGGGG
CATCTGATCAAACAATGTATATGAAGAAGAAGGTCATACTTATTTTTAT
CTTTTCTCTACTAAACTAAGTGATTTTCATGAATGTCTTATTCATATCT
TTTATTTCCAGTAAGACTTCTGTTTCATTTTAGCATTTCTTCTATTTGT
TCCATTGATCCAGATGTTTTCTTGGATTGACTAGATTGTTTCTTCTTTA
CTCAGTGTTCATCCCCACATGCCTAATGTTATCTCTCCTTTGTCCCACA
TAATTTTAGAGATCGTACACTATGCTTATTTTCTCTACAGAAGAACTGT
GTACTTATCTCTTGGTGATTTACCAGGTGTGGAGGTTCTCTTGCCCAGT
CCCCCACTTTAGACTACTTTCTCCATG (SEQ ID NO 9)
>BC_SSM_236_PW_1 (SET F)
GACCTATCCCTTTCCTATTTGGTTTCATTTGATCCAGGATGTTTTCTTG
GATTGACTAGATTGTTTCTTCTTTACTCAGTGTTCATCCCCACATGCCT
AATGTTATCTCTCCTTTGTCCCACATAATTTTAGAGATCGTACACTATG
CTTATTTTCTCTACAGAGAACTGTGTACTTATCTCTTGGTGATTTACCA
GGGTGTGAGGTTCTCTTGCCCAGTCCCCACTTTAGACTACTTCTCCTTG
TAGCATAAAATAAAATACATATTGTTTTTGACAAAAATGTATTAAATGG
TTTGTTTTGTTGCATTTTTATAAGTTCTTGAAAAAAAATGTTCTTGCCA
TCAACATCTCTTAACACACATCTTCAATCCCAGATTTGCAAACTAAATT
AAGAAATTTTTTCTATATTTTTCTCATGGATGCTAATTCTTAGTTTGTT
TTACTTTATCCAAGATGTCTATAAACTTCTAAATAAATTTTTATTGTCA
AGACTTCAAAAAAGAGTATAATACTCAGAGTTCATTACATGTAGGGAGG
ATATTATTATATTTTGTTTATTTTATAATTAGCATTTTATATGCCAATT
TTTAAGCCATTTCATTACTGACTACTATACTTTGAGCTATAATTTCTGT
CAGATCACATTTTTCATTGTTTTTTTGTTTCCTGGTACTCCCTATCCAA
CCTATAATTTTTTTTACAAAATAAAATACTTTCTCTTTAAATTTACTTC
TGTATTTCCATGTATTTATTTTAATATTTTTGAAAATATTGTAGATAAA
TGATAAACTTGAAATTTGGGATGTCTGAGCAATTTTGAATAAAATATAC
TCTTCTTTTATAAAACACCCTTCAAATAGCAGATGTTATCCTAACCAAC
CCTCATTATCTTTTTTTAGTTAATTAATTTTAACCTTACAATATTGTTA
TGCTTTGCCAAATATTGAATGGATTCCGACACAGGTTACACGTGATGCC
ATCCTGGAAGCATCTACGTCTGCCTTCCTAATACCATCT
(SEQ ID NO 10)

Complete Sequence of buCSN2 Promoter Containing Exon1, Intron1 and Exon2

(SEQ ID NO 11)
TCTGGTCCAATCGAATCCATCTCTATCAATTAATATAATTCAAAATTGG
TGAGAGACAGTCTTAAGGAAATGCTGTGTTTATTGCACAATATGTAAAG
CATCTTCCTGAGAAAAGGGAAATGTTGAATGGGAAGGACATGCTTTATT
TTGTATTCCTTTTCTCAGAAATCACACTTTTTTGCCTGTGGCCTTGGCA
ACCAAAAGCTAACACATAAAAAAAGGCATATGAAGTAGCCAAGGCCTTT
TCTAGTTATATCTATGACATTGAGTTCATTTTATCATTTATTTTCCTGA
CTTCCTCCTGGGTCCATATGAGAATCTTAGAATGAATATTAGCTGAAAA
TCCAAATACATAGTAGATGTTGATTTGGGTTTTCTAAGCAATACAAGAC
TTGTATGACAGTGAGATGTATTACCATCCAACACACATCTCAGCATGAT
ATAAACGTAAGGTATATTGTGAAGAAAAATGATCAATTATGTCAAAGTG
CTTACTTTAGAAGATCATCTATCTGTCCCAAAGCTGTGAATATATATAT
TGAATGTAATGAATAGATGAAACAAACCTTGTAAAAATGAGTAGTGTAA
AATACAACTACATTTATGAACATCTATCACTAAAGAGGCAAAGAAAGTT
GAGGACTGCTTTTGTAATTGGGCTCCTATTAATAAAAAGTACTTCTGAG
GTCTGGCTTAGACTCTATTGTAGTACTTATGGTAAGACCCTCCTCCTGT
CTGGGCTTTCATTTTCTTTCTTCCTTCCCTCATTTGCCCTTCCATGAAT
ACTAGCTGATAAACATTGGCTCACTATAAAAGATATGAGGCCAAACTTG
AGCTGTCCCATTTTAATAAATCTGTATAAATAATATTTGTTCTACAGAA
GTATTCTCTAAATAAATGTTACTTTCTCTCTTAAAATCCCTCAACAAAT
CCCCACTATCTAGAGAATAAGATTGGCATTCCCTGGAGTCACAGCATGC
TTTGTCTGCCATTATCTGACCCCTTTCTCTTTCTCTCTTCTCACCTCCA
TCTACTCCTTTTTCCTTGCAATTCATGACCCAGATTCACTGTTTGATTT
GGCTTACATGTGTGTGTGCTGAGTTGTGTCTCACTCTTGTCAACCCCAT
GAATGATAGTCCACCAGGCTCCAGTGTCCATGAAATTTTCCAGTCAAGA
ATGCTGGAGTGGATTGCATTTCCTACTCCATTTGATTAATTTAGTGACT
TTTAATTTTTTTTTCCCTCCATATTCAGGAGGCTATTCTTTCCTTTTAG
TCTATACTGTCTTTGCTCTTCAGGTCTAAGCTATCATCATGTGCTTGTT
AGCTTGTTTCTTTCTCCATTATAGCATAACACTAACAACTATTCAGGTT
AGCATGAGATTGTGTTCTTTGTGTGGCCTGTGTATTTTCTGGGTGGTGT
ATTAGAATTACCCTAGATCTCAAAGGACCCACCGAATACTAAAGAAGAC
CTCATTGTGGTTACATATTTGGGGACTCGGTCAAAACTTCGCATATCCA
GCAAAGTCTGTAGCTACTGGACAATTTAATTTCCTTTATCAGATTGTGA
ATTATTCCCTTTAAAATGCTCCCCAGAATTTCTGGGGACAGAAAAATAG
GAAGAATTCATTTTCTAATCATGCAGATTTCTAGGAATTCAAATTCACT
ATTGGYTTTATTTCAAACCACAAAATTAGCATGCCATTAAATACTATAT
ATAAACAGCCACAAAATCAGATCATTATCCATTCAGCTCCTCCTTCACT
TCTTGTCCTCTACTTTGGAAAAAAGGTAAGAATCTCATATATAATTTCA
GTGTATCTGCTACTCATCTTTATTTCAGACTAGGTTAAAATGTAGAAAG
AACATAATTGCTTAAAATAGATCTTAAAAATAAGGGTGTTTAAGATAAG
GTTTACACTATTTTCAGCAAATTTGTTAAAAAATAGAAGCAACTATAAA
GATTTGATAAAAATTATAGTGACTGCAAATGTTTTAGGAATATAATAAG
ATGTAGTAACAGTGGTTGCTATTTTCTTTACCACGAGACTAGTTAACAG
GCTGTATTAAAAGATCTTTTCTTGAATTAAATATTTTCAATTTGATTAA
ACCTATCTCAGCCATAAAGGCAAGCACATTTAATTTATACTATGGGGAT
TTGAAATAATTATTACTGAAGAAGCTCTACCAAACAAAAAGTTTATAGA
GCTAGCAGAGGGAGAGGGTGGGATGATTTGGGGAGAATGACATTGAAAC
ATGTAATAATATCATGTATGAAATGAGTCGCCAGTCCACGTTCGATGCA
CGATACTGGATGCTTGGGGCTGGTGCACTGGGACGACCCAGAGGGATGG
TATGGGGAGGGAGGAGGGAGGAGGGTTCAGGATGGGGAACACGTGTATA
CCTGTGGCGGATTCATTTCAATATTTGGCAAAACCAATACAATATTGTA
AAGTTTAAAAATAAAATAAAATTAAAAAAAAAGATAAAGAGGGTTGTTA
GGATACATCTGCTATTTGAAAGGTGTTTATAAAAGAAGAGTATATTTAT
CAAAATTGCTCAGAACATCCAAATTTCAAGTTTATCATTTATCTTACAA
TATTTCAAAAATATTAAAATAAATACATGAAATACAGAAGTAAATTAAA
GAGAAAGTATTTTATTTTGTAAAAAAAATTATAGGTTGGATAGGGAGTA
CCAGGAAACAAAAAACAATGAAAAATGTGATCTGACAGAAATTATAGCT
CAAAGTATAGTAGTCAGTAATGAAATGGCTTAAAAATTGGCATATAAAA
TGCTAATTATAAAATAAACAAAATATAATAATATCCTCCCTACATGTAA
TGAACTCTGAGTATTATACTCTTTTTTGAAGTCTTGAAATAAAAATTTA
TTTAGAAGTTTATAGACATCTTGGATAAAGTAAAACAAACTAAGAATTA
GCATCCATGAGAAAAATATAGAAAAAATTTCTTAATTTAGTTTGCAAAT
CTGGGATTGAAGATGTGTGTTAAGAGATGTTGATGGCAAGAACATTTTT
TTTCAAGAACTTATAAAAATGCAACAAAACAAACCATTTAATACATTTT
TGTCAAAAACAATATGTATTTTATTTTATGCTACAAGGAGAAGTAGTCT
AAAGTGGGGACTGGGCAAGAGAACCTCACACCCTGGTAAATCACCAAGA
GATAAGTACACAGTTCTCTGTAGAGAAAATAAGCATAGTGTACGATCTC
TAAAATTATGTGGGACAAAGGAGAGATAACATTAGGCATGTGGGGATGA
ACACTGAGTAAAGAAGAAACAATCTAGTCAATCCAAGAAAACATCTGGA
TCAATGGAACAAATAGAAGAAATGCTAAAATGAAACAGAAGTCTTACTG
GAAATAAAAGATATGAATAAGACATTCATGAAAATCACTTAGTTTAGTA
GAGAAAAGATAAAAATAAGTATGACCTTCTTCTTCATATACATTGTTTG
ATCAGATGCCCCTCAATAAAACTGAGTCTCCAATAGAACTGAAACCTTA
ATATTTTGTTCACTGCTCTAATCCCAGAATCTAAGCAATATCTGGCAAT
AAAAATTAATAAATAAATATTTTTTAATAAGTGAATCAATCACTTAATT
TTTCTGTAAATATCTGTAACTTCTGTCTTTCCAAAAAACACTCATAAGT
ACTGTGAATAAGATGAAAAAGAGTGGAATAAGATATAGGCTGTTAGCTG
AAAACATCTGAATGGCTGGCAGTGAAACATTAACTTGAAATGTAAGATT
AATGAGTAATAGTAAATTTTAACCTTGGCTATATGATAAAATGTCTATT
AATATTTTTCTAAAATACAGGGCTTTTTGTTTTTGCCATGAGGTTTGCA
GGATCTTGGTTCCCTGACGAGGGATCAAACCTGCGCTCCCCTGGAAGCA
TGGAGTCTTGGACATTTGTATTATACACTATCTTTGGTTTCTTTTAAAG
GGAAGTAATTCTACTTAAATAAGAAAATAGATTGACAAGTAATACACTG
TTTCCTCATCTTCCCATTCACAGAAATTGAGAGCCATGAAGGTCCTCAT
CCTTGCCTGCCTGGTGGCTCTGGCCCTTGCAAGAGAGGTAAATA

We claim:

1. An in vitro method of isolating and cloning a specific promoter sequence of buffalo β-caesin containing a full length buCSN2 Promoter along with Exon1, Intron1 and Exon2, said method comprising the steps of:
(i) isolating from the blood of buffalo genomic fragments comprising buCSN2 (buffalo β-caesin) promoter region and regions of exon1, intron1 and exon2;
(ii) cloning the isolated genomic fragment into pCMV-SPORT6 vector to develop a pCMV-SPORT6-buCSN2;
(iii) further sub cloning buCSN2 from pCMV-SPORT6-buCSN2 into pIRES2-EGFP vector to develop the pbuCSN2-IRES2-EGFP plasmid.
(iv) sequencing the clones of step (iii)
(v) transfecting the linearized DNA construct of step (iii) into cell line derived from the mammary gland of said buffalo.
(vi) adding regulatory sequences to buCSN2 for targeting protein to specific compartments of cell or for enhanced secretory activity.

2. The method of claim 1, wherein the said buffalo β-caesin promoter drives the expression of a foreign gene inserted in a 5′→3′ orientation and is transfected in mammary gland cells of said buffalo.

3. The method of claim 2, wherein the said foreign gene is any therapeutic or recombinant protein including insulin, growth hormones, growth hormone releasing factors, somatostatin, tissue plasminogen activators, tumor necrosis factors, lipocortin, coagulation factors VIII and IX, interferons, colony stimulating factor, the interleukins and urokinase.

4. The method of claim 1, wherein the linearised fragment of said DNA containing the BuCSN2 promoter region is used to transfect mammalian tissue of buffalo udder cells directly in vivo expressing such therapeutic proteins in their milk.

5. The method of claim 1, wherein the linearised fragment of the DNA containing genes cloned under the buCSN2 promoter region is used to generate transgenic buffaloes expressing therapeutic proteins in their milk.

6. The method of claim 1, wherein the DNA containing genes cloned under the buCSN2 promoter region is used to transfect mammalian tissue of buffalo udder cells directly for providing resistance to microbes causing mastitis in the udder gland thereby rendering the animal disease free.

7. The method of claim 1, wherein the DNA containing genes cloned under the buCSN2 promoter region is used to generate transgenic buffaloes expressing genes which provide resistance to microbes other than the udder gland thereby rendering the animal substantially free of diseases.

8. The method of claim 1, wherein the linearised fragment of the DNA containing genes cloned under the buCSN2 promoter region is used to generate other transgenic mammals such as large animals except human beings by expressing such therapeutic proteins in their milk.

9. The method of claim 8, wherein the mammal is preferably a buffalo (Bubalus bubalis) that produce large quantities of milk.


SEQUENCE LISTING (11 sequences)

SEQ ID NO: 1 - DNA, 19 bp, Artificial sequence
  synthetic sequence
gatttccaagtctccaccc

SEQ ID NO: 2 - DNA, 20 bp, Artificial sequence
  synthetic sequence
atatagacaaacgcacaccg

SEQ ID NO: 3 - DNA, 31 bp, Artificial sequence
  synthetic sequence
gcctgcagtctggtccaatcgaatccatctc

SEQ ID NO: 4 - DNA, 31 bp, Artificial sequence
  synthetic sequence
gccccgggtatttacctctcttgcaagggcc

SEQ ID NO: 5 - DNA, 1085 bp, Artificial sequence
  synthetic sequence
gagctgggggtagtttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtctggtccaatcgaatccatctctatcaattaatataattcaaaattggtgagagacagtcttaaggaaatgctgtgtttattgcacaatatgtaaagcatcttcctgagaaaagggaaatgttgaatgggaaggacatgctttattttgtattccttttctcagaaatcacacttttttgcctgtggccttggcaaccaaaagctaacacataaaaaaaggcatatgaagtagccaaggccttttctagttatatctatgacattgagttcattttatcatttattttcctgacttcctcctgggtccatatgagaatcttagaatgaatattagctgaaaatccaaatacatagtagatgttgatttgggttttctaagcaatacaagacttgtatgacagtgagatgtattaccatccaacacacatctcagcatgatataaacgtaaggtatattgtgaagaaaaatgatcaattatgtcaaagtgcttactttagaagatcatctatctgtcccaaagctgtgaatatatatattgaatgtaatgaatagatgaaacaaaccttgtaaaaatgagtagtgtaaaatacaactacatttatgaacatctatcactaaagaggcaaagaaagttgaggactgcttttgtaattgggctcctattaataaaaagtacttctgaggtctggcttagactctattgtagtacttatggtaagaccctcctcctgtctgggctttcattttcttcttcctttccctcatttgcccttccatgaatactagctgataaacattggctcactataaagatatgagcaaactgagctgtccatttataaatctgtataaaataaatattttgttcttacaggaagtattttcctctta

SEQ ID NO: 6 - DNA, 980 bp, Artificial sequence
  synthetic sequence
tattgacccctttgttttgggcctccttattaataaaaagtacttctgaggtctggcttagactctattgtagtacttatggtaagaccctcctcctgtctgggctttcattttctttcttccttccctcatttgcccttccatgaatactagctgataaacattggctcactataaaagatatgaggccaaacttgagctgtcccattttaataaatctgtataaataatatttgttctacagaagtattctctaaataaatgttactttctctcttaaaatccctcaacaaatccccactatctagagaataagattggcattccctggagtcacagcatgctttgtctgccattatctgacccctttctctttctctcttctcacctccatctactcctttttccttgcaattcatgacccagattcactgtttgatttggcttacatgtgtgtgtgctgagttgtgtctcactcttgtcaaccccatgaatgatagtccaccaggctccagtgtccatgaaattttccagtcaagaatgctggagtggattgcatttcctactccatttgattaatttagtgacttttaatttttttttccctccatattcaggaggctattctttccttttagtctatactgtctttgctcttcaggtctaagctatcatcatgtgcttgttagcttgtttctttctccattatagcataacactaacaactattcaggttagcatgagattgtgttctttgtgtggcctgtgtattttctgggtggtgtattagaattaccctagatctcaaaggacccaccgaatactaaagaagacctcattgtggttacatatttggggactcggtcaaaacttcgcatatccagcaaagtctgtagctactggacaaattgactttccatttatcgaattgtgactcatccattgaaatgcctcctgatcggaacgagaatacggaggac

SEQ ID NO: 7 - DNA, 993 bp, Artificial sequence
  synthetic sequence
aaaaccatcaagacccccacccgaatactaaaagagacctcattgtggttacaataatttggggactcggccaaaacttccgcatatcccagccaaggtctgtagctactggacaatttaatttcctttatcagattgtgaattattccctttaaaatgctccccagaatttctggggacagaaaaataggaagaattcattttctaatcatgcagatttctaggaattcaaattcactattggttttatttcaaaccacaaaattagcatgccattaaatactatatataaacagccacaaaatcagatcattatccattcagctcctccttcacttcttgtcctctactttggaaaaaaggtaagaatctcatatataatttcagtgtatctgctactcatctttatttcagactaggttaaaatgtagaaagaacataattgcttaaaatagatcttaaaaataagggtgtttaagataaggtttacactattttcagcaaatttgttaaaaaatagaagcaactataaagatttgataaaaattatagtgactgcaaatgttttaggaatataataagatgtagtaacagtggttgctattttctttaccacgagactagttaacaggctgtattaaaagatcttttcttgaattaaatattttcaatttgattaaacctatctcagccataaaggcaagcacatttaatttatactatggggatttgaaataattattactgaagaagctctaccaaacaaaaagtttatagagctagcagagggagagggtgggatgatttggggagaatgacattgaaacatgtaataatatcatgtatgaaatgagtcgccagtccacgttcgatgccacgatactgcatgctggggctgtgccactggacgaccagaggatgtatggagagagggaaagaaggttccagaatggaaccgttatacccgtgcggaatcatcctaaattggcaaac

SEQ ID NO: 8 - DNA, 1009 bp, Artificial sequence
  synthetic sequence
ggtacccggttatttattcatggtatgaatgagtcgccagtccacgttcgatgcacgatactggatgcttggggctggtgcactgggacgacccagagggatggtatggggagggaggagggaggagggttcaggatggggaacacgtgtatacctgtggcggattcatttcaatatttggcaaaaccaatacaatattgtaaagtttaaaaataaaataaaattaaaaaaaaagataaagagggttgttaggatacatctgctatttgaaaggtgtttataaaagaagagtatatttatcaaaattgctcagaacatccaaatttcaagtttatcatttatcttacaatatttcaaaaatattaaaataaatacatgaaatacagaagtaaattaaagagaaagtattttattttgtaaaaaaaattataggttggatagggagtaccaggaaacaaaaaacaatgaaaaatgtgatctgacagaaattatagctcaaagtatagtagtcagtaatgaaatggcttaaaaattggcatataaaatgctaattataaaataaacaaaatataataatatcctccctacatgtaatgaactctgagtattatactctttttttgaagtcttgacaataaaaatttatttaagaagtttatagacatcttggataaagtaaaacaaactaagaattagcatccatgagaaaaatatagaaaaaatttctttaatttagtttgcaaaatctgggattgaagaatgtgtgttaagcagatgtttgatgggcaaggaaccatttttgttttcaaagaagctgtatgaaaaatgcaacaacaacctattttaattacattttgtcaaaaaacaatatgtattttaatttatgcctaacagggagaaagttagtctaagtggggactggcaagagaaatcctcacacccctggtaatcacaagagaaatagttacaccagtctctgtaagataggctaagttacgattctcttaca

SEQ ID NO: 9 - DNA, 1056 bp, Artificial sequence
  synthetic sequence
ctttagccggcttcgggccagtaaacgttaggggggggggagggagaggggcggatcccgggtatttacctctcttgcaagggccagagccaccaggcaggcaaggatgaggaccttcatggctctcaatttctgtgaatgggaagatgaggaaacagtgtattacttgtcaatctattttcttatttaagtagaattacttccctttaaaagaaaccaaagatagtgtataatacaaatgtccaagactccatgcttccaggggagcgcaggtttgatccctcgtcagggaaccaagatcctgcaaacctcatggcaaaaacaaaaagccctgtattttagaaaaatattaatagacattttatcatatagccaaggttaaaatttactattactcattaatcttacatttcaagttaatgtttcactgccagccattcagatgttttcagctaacagcctatatcttattccactctttttcatcttattcacagtacttatgagtgttttttggaaagacagaagttacagatatttacagaaaaattaagtgattgattcacttattaaaaaatatttatttattaatttttattgccagatattgcttagattctgggattagagcagtgaacaaaatattaaggtttcagttctattggagactcagttttattgaggggcatctgatcaaacaatgtatatgaagaagaaggtcatacttatttttatcttttctctactaaactaagtgattttcatgaatgtcttattcatatcttttatttccagtaagacttctgtttcattttagcatttcttctatttgttccattgatccagatgttttcttggattgactagattgtttcttctttactcagtgttcatccccacatgcctaatgttatctctcctttgtcccacataattttagagatcgtacactatgcttattttctctacagaagaactgtgtacttatctcttggtgatttaccaggtgtggaggttctcttgcccagtcccccactttagactactttctccatg

SEQ ID NO: 10 - DNA, 1020 bp, Artificial sequence
  synthetic sequence
gacctatccctttcctatttggtttcatttgatccaggatgttttcttggattgactagattgtttcttctttactcagtgttcatccccacatgcctaatgttatctctcctttgtcccacataattttagagatcgtacactatgcttattttctctacagagaactgtgtacttatctcttggtgatttaccagggtgtgaggttctcttgcccagtccccactttagactacttctccttgtagcataaaataaaatacatattgtttttgacaaaaatgtattaaatggtttgttttgttgcatttttataagttcttgaaaaaaaatgttcttgccatcaacatctcttaacacacatcttcaatcccagatttgcaaactaaattaagaaattttttctatatttttctcatggatgctaattcttagtttgttttactttatccaagatgtctataaacttctaaataaatttttattgtcaagacttcaaaaaagagtataatactcagagttcattacatgtagggaggatattattatattttgtttattttataattagcattttatatgccaatttttaagccatttcattactgactactatactttgagctataatttctgtcagatcacatttttcattgtttttttgtttcctggtactccctatccaacctataatttttttttacaaaataaaatactttctctttaaatttacttctgtatttccatgtatttattttaatatttttgaaaatattgtagataaatgataaacttgaaatttgggatgtctgagcaattttgaataaaatatactcttcttttataaaacacccttcaaatagcagatgttatcctaaccaaccctcattatctttttttagttaattaattttaaccttacaatattgttatgctttgccaaatattgaatggattccgacacaggttacacgtgatgccatcctggaagcatctacgtctgccttcctaataccatct

SEQ ID NO: 11 - DNA, 4112 bp, Artificial sequence
  synthetic sequence
tctggtccaatcgaatccatctctatcaattaatataattcaaaattggtgagagacagtcttaaggaaatgctgtgtttattgcacaatatgtaaagcatcttcctgagaaaagggaaatgttgaatgggaaggacatgctttattttgtattccttttctcagaaatcacacttttttgcctgtggccttggcaaccaaaagctaacacataaaaaaaggcatatgaagtagccaaggccttttctagttatatctatgacattgagttcattttatcatttattttcctgacttcctcctgggtccatatgagaatcttagaatgaatattagctgaaaatccaaatacatagtagatgttgatttgggttttctaagcaatacaagacttgtatgacagtgagatgtattaccatccaacacacatctcagcatgatataaacgtaaggtatattgtgaagaaaaatgatcaattatgtcaaagtgcttactttagaagatcatctatctgtcccaaagctgtgaatatatatattgaatgtaatgaatagatgaaacaaaccttgtaaaaatgagtagtgtaaaatacaactacatttatgaacatctatcactaaagaggcaaagaaagttgaggactgcttttgtaattgggctcctattaataaaaagtacttctgaggtctggcttagactctattgtagtacttatggtaagaccctcctcctgtctgggctttcattttctttcttccttccctcatttgcccttccatgaatactagctgataaacattggctcactataaaagatatgaggccaaacttgagctgtcccattttaataaatctgtataaataatatttgttctacagaagtattctctaaataaatgttactttctctcttaaaatccctcaacaaatccccactatctagagaataagattggcattccctggagtcacagcatgctttgtctgccattatctgacccctttctctttctctcttctcacctccatctactcctttttccttgcaattcatgacccagattcactgtttgatttggcttacatgtgtgtgtgctgagttgtgtctcactcttgtcaaccccatgaatgatagtccaccaggctccagtgtccatgaaattttccagtcaagaatgctggagtggattgcatttcctactccatttgattaatttagtgacttttaatttttttttccctccatattcaggaggctattctttccttttagtctatactgtctttgctcttcaggtctaagctatcatcatgtgcttgttagcttgtttctttctccattatagcataacactaacaactattcaggttagcatgagattgtgttctttgtgtggcctgtgtattttctgggtggtgtattagaattaccctagatctcaaaggacccaccgaatactaaagaagacctcattgtggttacatatttggggactcggtcaaaacttcgcatatccagcaaagtctgtagctactggacaatttaatttcctttatcagattgtgaattattccctttaaaatgctccccagaatttctggggacagaaaaataggaagaattcattttctaatcatgcagatttctaggaattcaaattcactattggttttatttcaaaccacaaaattagcatgccattaaatactatatataaacagccacaaaatcagatcattatccattcagctcctccttcacttcttgtcctctactttggaaaaaaggtaagaatctcatatataatttcagtgtatctgctactcatctttatttcagactaggttaaaatgtagaaagaacataattgcttaaaatagatcttaaaaataagggtgtttaagataaggtttacactattttcagcaaatttgttaaaaaatagaagcaactataaagatttgataaaaattatagtgactgcaaatgttttaggaatataataagatgtagtaacagtggttgctattttctttaccacgagactagttaacaggctgtattaaaagatcttttcttgaattaaatattttcaatttgattaaacctatctcagccataaaggcaagcacatttaatttatactatggggatttgaaataattattactgaagaagctctaccaaacaaaaagtttatagagctagcagagggagagggtgggatgatttggggagaatgacattgaaacatgtaataatatcatgtatgaaatgagtcgccagtccacgttcgatgcacgatactggatgcttggggctggtgcactgggacgacccagagggatggtatggggagggaggagggaggagggttcaggatggggaacacgtgtatacctgtggcggattcatttcaatatttggcaaaaccaatacaatattgtaaagtttaaaaataaaataaaattaaaaaaaaagataaagagggttgttaggatacatctgctatttgaaaggtgtttataaaagaagagtatatttatcaaaattgctcagaacatccaaatttcaagtttatcatttatcttacaatatttcaaaaatattaaaataaatacatgaaatacagaagtaaattaaagagaaagtattttattttgtaaaaaaaattataggttggatagggagtaccaggaaacaaaaaacaatgaaaaatgtgatctgacagaaattatagctcaaagtatagtagtcagtaatgaaatggcttaaaaattggcatataaaatgctaattataaaataaacaaaatataataatatcctccctacatgtaatgaactctgagtattatactcttttttgaagtcttgacaataaaaatttatttagaagtttatagacatcttggataaagtaaaacaaactaagaattagcatccatgagaaaaatatagaaaaaatttcttaatttagtttgcaaatctgggattgaagatgtgtgttaagagatgttgatggcaagaacattttttttcaagaacttataaaaatgcaacaaaacaaaccatttaatacatttttgtcaaaaacaatatgtattttattttatgctacaaggagaagtagtctaaagtggggactgggcaagagaacctcacaccctggtaaatcaccaagagataagtacacagttctctgtagagaaaataagcatagtgtacgatctctaaaattatgtgggacaaaggagagataacattaggcatgtggggatgaacactgagtaaagaagaaacaatctagtcaatccaagaaaacatctggatcaatggaacaaatagaagaaatgctaaaatgaaacagaagtcttactggaaataaaagatatgaataagacattcatgaaaatcacttagtttagtagagaaaagataaaaataagtatgaccttcttcttcatatacattgtttgatcagatgcccctcaataaaactgagtctccaatagaactgaaaccttaatattttgttcactgctctaatcccagaatctaagcaatatctggcaataaaaattaataaataaatattttttaataagtgaatcaatcacttaatttttctgtaaatatctgtaacttctgtctttccaaaaaacactcataagtactgtgaataagatgaaaaagagtggaataagatataggctgttagctgaaaacatctgaatggctggcagtgaaacattaacttgaaatgtaagattaatgagtaatagtaaattttaaccttggctatatgataaaatgtctattaatatttttctaaaatacagggctttttgtttttgccatgaggtttgcaggatcttggttccctgacgagggatcaaacctgcgctcccctggaagcatggagtcttggacatttgtattatacactatctttggtttcttttaaagggaagtaattctacttaaataagaaaatagattgacaagtaatacactgtttcctcatcttcccattcacagaaattgagagccatgaaggtcctcatccttgcctgcctggtggctctggcccttgcaagagaggtaaata